(19) United States
(12) Patent Application Publication
(10) Pub. No.: US 2001/0022980 A1
(43) Pub. Date: Sep. 20, 2001

(54) Dietary supplement for individuals under stress

(75) Inventors: Stacey J. Bell, Belmont, MA (US); R. Armour Forse, Brookline, MA (US); Bruce R. Bistrian, Ipswich, MA (US)

Correspondence Address: Alice O. Carroll, Esq., HAMILTON, BROOK, SMITH & REYNOLDS, P.C., Two Militia Drive, Lexington, MA, 02421-4799, US

(73) Assignee: Beth Israel Deaconess Medical Center, Boston, MA (US)

(21) Appl. No.: 09/730,520

(22) Filed: Dec. 5, 2000

Related U.S. Application Data
(63) Continuation of application No. 09/100,690, filed on Jun. 19, 1998, now abandoned.

(51) Int. Cl. A61K 35/78; A61K 31/718; A61K 31/715; A61K 31/7016; A61K 31/522; A61K 31/685

(52) U.S. Cl. 424/771; 424/768; 514/53; 514/58; 514/60; 514/264; 514/78; 514/561

(57) ABSTRACT

Described herein is a nutritional supplement which provides fats, proteins and carbohydrates of compositions and proportions so as to have the effects of reducing the symptoms of stress and improving performance by causing a reduction in the level of PGE₂. Of particular importance is a balance of carbohydrate, proteins and fats derived from different structural families.

### RELATED APPLICATION

This application is a continuation of application Ser. No. 09/100,690, filed Jun. 19, 1998. The entire teachings are incorporated herein by reference.

### BACKGROUND OF THE INVENTION

Healthy people living in this fast-paced society are subjected to many sources of stress related to their job, family, money issues and the like. Full time students and employees, and especially the business traveler are highly stressed. For the stressed individual, there is a constant fear of not performing at his or her mental or physical best from lack of sleep and exercise, and from improper diet.

An agency of the United States Government has expressed concern that the armed forces are not performing at their mental and physical best while under stress. At the request of the Assistant Surgeon General of the Army, the Food and Nutrition Board of the National Academy of Sciences set up a committee to advise the U.S. Department of Defense on improving performance of the military under stress. The Committee on Military Nutrition Research (CMNR) was formed and instructed to identify nutritional factors that may influence the physical and mental performance of the military under all environmental extremes. This Committee stated that “combat settings do not differ from those in a regular workplace” insofar as the effects from stress are concerned. A dietary supplement that can enhance the performance of stressed civilians as well as military personnel is desirable.

### SUMMARY OF THE INVENTION

Described herein is a dietary supplement whose ingredients, when ingested, improve the ability of an individual to function under stressful conditions. The dietary supplement comprises fats and oils provided by borage oil, fish oil, medium-chain triglycerides, and other oils from plant sources, carbohydrate (simple sugars, oligosaccharides, complex carbohydrates), protein, choline, glutamine and creatine. Also described herein are methods for improving the ability of an individual to function in stressful situations, comprising administering to the individual the dietary supplement described herein.

### DETAILED DESCRIPTION OF THE INVENTION

The invention relates to a dietary supplement comprising ingredients formulated to supply to a person under stress nutrients that can alleviate the symptoms of stress. These nutrients are likely to be in short supply in the diet of a person under stress, as poor eating habits often result from stressful situations. Although the dietary supplement of the invention is intended primarily for humans, it can also be used as a supplement to the diet of other mammals, especially other primates, as animals (racehorses, for example) are also subject to stress, although the sources and symptoms of stress may be different.

A number of nutrients were reviewed by the CMNR for their impact on performance (Committee on Military Nutrition Research: Food and Nutrition Board, Institute of Medicine. Food Components to Enhance Performance, (B. M. Marriott, ed.) National Academy Press, Washington D.C., 1994). Several nutrients were found to have effects beneficial to stressed individuals.

It is known that aspirin and ibuprofen can alleviate some of the symptoms of stress such as headaches, muscle aches, and poor quality sleep, because these products are effective in reducing the production of cytokines and prostaglandin E₂ (PGE₂). The cytokines whose synthesis is reduced with aspirin or ibuprofen are the interleukins IL-1, IL-6 and tumor necrosis factor (TNF). This not only reduces the symptoms of stress, but also improves performance because of improved appetite, decreased loss of muscle tissue, and less wasteful burning of calories.

Different fats allow for biosynthesis of eicosanoids of different families (W. G. Linscheer and A. J. Vergroesen, Chapter 3, pages 47-88 In: Shils M. E., Olson J. A., Shike M. Modern Nutrition in Health and Disease, Philadelphia: Lea & Febiger, 8^{th} ed., 1994). The dietary supplement of the invention is formulated to supply linolenic acid and omega-3 fatty acids, among other fatty acids. Linolenic acid and omega-3 fatty acids become incorporated into the body's white blood cells. This causes a reduction in the production of PGE₂ and the cytokines IL-1, IL-6 and TNF, which will lessen symptoms associated with stress and improve performance.

The goal is to decrease the production of pro-inflammatory prostanoids of the “2 series” and leukotrienes of the “4 series” and increase production of prostanoids of the “1 and 3 series” and leukotrienes of the “5 series.”

A good ratio to suppress inflammation is 1/1.8 EPA/GLA (for instance, by using 240 mg/450 mg in patients with rheumatoid arthritis (J. J. Belch et al., Ann. Rheum. Dis. 47:96-104 (1988)). GLA increases the production of dihomo-gamma-linolenic acid (DGLA), increasing the ratio of DGLA/AA, driving the favorable production of eicosanoids. Appropriate selection of EPA, GLA and ALA further guarantees that the DGLA will not produce more AA, thereby decreasing PGE₂. (EPA=eicosapentaenoic acid; GLA=γ-linoleic acid; AA=arachidonic acid; ALA=α-linolenic acid).

Changes in the composition of the white blood cells can be confirmed by analysis of the white blood cells for fatty acid content, and for the amount of the cytokines (IL-1, IL-6 and TNF) and PGE₂ produced by the white blood cells. Blood samples can be taken prior to starting a diet including the dietary supplement of the invention, after three weeks, and after six weeks. After six weeks on a diet containing 10 grams per day of fat from fish oil, a significant increase in the omega-3 fatty acid content of the white blood cells has been observed in patients infected with HIV (Bell, S. J. et al., Journal of Parenteral and Enteral Nutrition 20:43-49 (1996)).

In addition to supplying the fatty acids to reduce PGE₂ biosynthesis, the dietary supplement also supplies a balance of carbohydrates that do not induce a high level of insulin, glycogen-sparing medium-chain triglycerides, high quality protein, choline for muscle and mental performance, and creatine for maintenance of muscle and strength. The combined effect of these nutrients is to alleviate the symptoms of stress.

Oils

Included in the dietary supplement are one or more oils rich in fatty acids which down-regulate PGE₂ production and up-regulate PGE₁ and PGE₃ production. Oils effective to this end are the oil of walnuts, fish oil from menhaden or salmon, flaxseed oil, evening primrose oil, borage oil, and possibly olive, canola or sesame oils which are rich in the omega-3 fatty acids. (See Forse et al., U.S. Pat. No. 5,762,935 on the anti-inflammatory and infection-protective effects of sesame oil; see Forse et al., U.S. Pat. No. 5,260,336 on the effects of consumption of olive oil.) Soybean oil and canola oil should be beneficial because they are similar to walnuts; they are both rich in linolenic acid and have less linoleic acid than other vegetable oils. Flaxseed oil has the highest ratio of α-linolenic acid to linoleic acid. A diet supplemented with flaxseed oil has produced measurable effects on cytokines, prostaglandins and TXA₂ (G. Caughey et al., American Journal of Clinical Nutrition 63:116-122 (1996)). Walnuts are rich in ALA (7 g per 100 g of nuts; 12% of the total fat). Walnuts are rich in monounsaturated fatty acids as well.

Omega-3 fatty acids from marine oils (EPA=C20:5, n-3 and DHA=C22:6, n-3) or from vegetable oils high in α-linolenic acid (e.g., flaxseed oil) lead to displacement of C18:2, n-6 and decreased production of pro-platelet aggregatory thromboxane A₂ (TXA₂), PGE₂ and prostaglandins of the “3 series.” Menhaden oil is approximately 14% EPA and 10% DHA (docosahexaenoic acid), but can vary in fatty acid content.

γ-linolenic (C18:3, n-6) from borage oil competes with AA as a substrate for oxidative enzyme systems to prevent the formation of inflammatory eicosanoids (e.g., PGE₂ and leukotriene B4 (LTB₄)). In human studies, significant reductions in PGE₂ and leukotrienes B4 and C4 produced by stimulated monocytes were seen after 12 weeks of borage seed oil supplementation to provide GLA (S. Pullman-Mooar et al., Arthritis Rheum. 33:1526-1533 (1990)). Borage oil is rich in GLA; 4.5 g of the oil contains about 0.55 g GLA.

Evening primrose oil contains one-half the amount of GLA that borage oil does, and both improved symptoms in patients with rheumatoid arthritis (M. Brzeski et al., Br. J. Rheumatol. 30:370-372 (1991); L. J. Leventhal, Ann. Intern. Med. 119:867-873 (1993)).

Blackcurrant seed oil has both GLA and ALA (G. A. Tate and R. B. Zurier, Agents Actions 43:35-38 (1994)). This decreases inflammation. In studies on rats, GLA suppressed the cellular phase of inflammation (polymorphonuclear leukocyte accumulation, phagocytosis, and lysosomal enzyme activity), while EPA suppressed the fluid phase (exudate volume and protein concentration) (G. A. Tate et al., Arthritis Rheum. 31:1543-1551 (1988)). Therefore, a diet combining plant seed oil (providing GLA) and fish oil (providing EPA; or flaxseed oil providing ALA) can provide alternative substrates for oxidative metabolism (other than arachidonic acid, a precursor of PGE₂ and LTB₄) and thereby reduce inflammation.

Linolenic acid can also be supplied by a variety of nuts and seeds, for example, walnuts, peanuts, cashews, pistachios, Brazil nuts, hazelnuts, pecans, almonds, etc. The nuts and oils included in the dietary supplement can be of a single type or can be a combination of several types.

It appears that it is more beneficial to ingest ALA and GLA together than GLA alone. The combination drives the formation of PGE₁. The addition of EPA with these two fats further guarantees that the di-homo-gamma-linolenic acid will not be converted to arachidonic acid and PGE₁ will be formed instead. Thus, it is optimal for a dietary supplement to contain ALA (e.g., from nuts or flaxseed oil), GLA (e.g., from borage oil), and EPA (e.g., from fish oil) to increase production of prostaglandins of the 1 and 3 series and decrease production of prostaglandin from the 2 series. See review discussing rationale and experimental data that led to clinical trials of γ-linolenic acid for the reduction of inflammation in rheumatoid arthritis: Rothman, D. et al, Semin. Arthritis Rheum. 25:87-96 (1995).

The American Heart Association and the Food Pyramid Guide for healthy eating recommend that Americans eat foods that contain less than 30% of their total kcal from fat. The dietary supplement of the invention contains a higher level of fat (up to 50% of total kcal from fat). However, the fats in the dietary supplement have been chosen for the specific objective of affecting the biosynthesis of prostaglandins, cytokines and leukotrienes as described above.

A further object of the dietary supplement is to keep insulin levels down, which is achieved in two ways in the dietary supplement. (1) Fats do not induce insulin production. (2) The carbohydrates of the dietary supplement have been selected to provide a balance of carbohydrates of various glycemic indices. Low insulin keeps tryptophan and serotonin levels down; this is beneficial in stress to prevent the avoidance of carbohydrate-rich foods at the next meal or snack. Carbohydrates are required for energy during stress, but ingesting too much carbohydrate having a high glycemic index increases tryptophan and serotonin levels, which decrease the desire to eat carbohydrates. The mixture of carbohydrates with varying glycemic indices overcomes this problem.

Medium-Chain Triglycerides (MCTs)

These fats are more rapidly absorbed and metabolized compared to conventional fats, which are long-chained triglycerides. The MCTs can spare the glucose stored in the muscle as glycogen, thereby enhancing endurance and physical and mental performance. Medium-chain fatty acids incorporated into triglycerides are usually defined as being those fatty acids 8-12 carbons long.

Carbohydrates

The glycemic index is the area under the curve in a plot of blood sugar measurements versus time, wherein the blood sugar measurements are taken over a period of time after a carbohydrate meal. Thus, the glycemic index of a carbohydrate is a relative measure of the rate and amount of glucose released into the blood from a carbohydrate. In a preferred embodiment, the dietary supplement contains a variety of carbohydrate sources, each source selected from a different glycemic index (see Modern Nutrition in Health and Disease, eighth edition, Lea & Febiger, publishers, 1986, especially Volume 2, page 1270 and Appendix page A-135), so that glucose is released sequentially into the blood as the dietary supplement is digested and absorbed.

In a preferred embodiment, the dietary supplement contains one or more carbohydrates having a low glycemic index (e.g., from less than about 70), one or more sources of carbohydrate having an intermediate glycemic index (e.g., from about 70 to about 80), and one or more sources of complex carbohydrate having a high glycemic index (e.g., from greater than about 90) and combinations of these. For example, the dietary supplement can contain sucrose, which appears in the blood first after ingestion; high fructose corn syrup, such as high fructose corn syrup comprising 42% fructose and about 43% glucose, which appears next; a source of oligosaccharide that can supply 4 kcal/gram, such as maltodextrin (soybean oligosaccharide, galactooligosaccharide, isomaltooligosaccharide or lactosucrose may also serve as sources of oligosaccharide) as found in corn syrup solids, which comprises glucose polymers and appears next; and uncooked cornstarch, which is slowest to release into the blood as glucose and lasts up to eight hours in the blood (i.e., having the lowest glycemic index). See Kaufman et al., U.S. Pat. No. 5,605,893 and U.S. Ser. No. 08/631,584.

Sucrose is the preferred simple carbohydrate (i.e., among carbohydrates of high glycemic index) because it provides the most desirable organoleptic properties compared to other sweeteners. Uncooked cornstarch is a preferred complex carbohydrate having a low glycemic index but should be included in food formulations or portions thereof which are not cooked or heat processed since the heat will break down the complex carbohydrate into simple carbohydrates, wherein simple carbohydrates are mono- or disaccharides, creating a high glycemic index product. Staggering the release of sugars into the body prevents too much of an exacerbation of catecholamine excretion occurring immediately after ingestion of the dietary supplement. Too much glucose released into the blood at once raises insulin and serotonin levels, which could decrease appetite or the desire for carbohydrate-rich foods at the next meal. Neither would benefit a stressed person who needs to eat regular meals containing all nutrients, including carbohydrate. The food bars preferably contain combinations of sources of carbohydrate of three levels of chain length, one or more sources of each level of chain length (e.g., sucrose, maltodextrins, and uncooked cornstarch) which are released sequentially into the blood, thus not causing a great rise in insulin or serotonin levels (Bell, S. J. and R. A. Forse. Timed-release glucose for patients with insulin-dependent diabetes. Submitted for publication to Diabetes Educator, 1998.).

Proteins

Consumption of foods containing a combination of both carbohydrate and protein has been shown to overcome fatigue and improve mood and performance better than those foods that are protein-free (e.g., candy bars). A preferred way to use the dietary supplement during stress is as an in-between-meal snack. The dietary supplement preferably contains at least 4% of the total calories from protein.

Sources of protein can be any suitable protein utilized in nutritional formulations and can include whey protein, whey protein concentrate, whey powder, egg, soy protein, soy protein isolate, caseinate (e.g., sodium caseinate, sodium calcium caseinate, calcium caseinate, potassium caseinate), animal and vegetable protein and mixtures thereof. When choosing a protein source, the biological value of the protein should be considered first, with the highest biological values being found in caseinate, whey, lactalbumin, egg albumin and whole egg proteins. In a preferred embodiment, the protein is a combination of whey protein concentrate and calcium caseinate. These proteins have high biological value; that is, they have a high proportion of the essential amino acids. See Modern Nutrition in Health and Disease, eighth edition, Lea & Febiger, publishers, 1986, especially Volume 1, pages 30-32.

Preferred protein sources are rich in the amino acids cysteine and tyrosine. Cysteine is a precursor for glutathione, which is an important cellular antioxidant. Glutathione is abundant in the cytoplasm, nuclei, and mitochondria. Glutathione can de-toxify both soluble and lipid peroxidases, which is important during stress. It is important to have sufficient quantities of this under stress to preserve the cell membranes. During stress, exercise, and nutrient limitation, the body has an increased need for glutathione.

TABLE 1

| Amino acid per 100 g | Whey | Egg Protein | Soy flour |
|---|---|---|---|
| cysteine | 2.2 g | 2.1 | 1.5 g (e.g., NutriSoy ™ 7B |
| | | | and TVP 165 and 163 |
| | | | series of ADM) |
| tyrosine | 4.3 g | 3.2 | 3.5 g (e.g., NutriSoy ™ 7B |
| | | | and TVP 165 and 163 |
| | | | series of ADM) |

Tyrosine is a precursor for the neurotransmitters dopamine, norepinephrine and epinephrine. These are required by the body to respond to stress. High-quality protein such as whey is a rich source of tyrosine and can be used as a protein source for the dietary supplement of the invention.

Cysteine and glutamine are both substrates for glutathione synthesis. During stress, more glutathione is required, (J. Thomas, pages 501-523 In: Shils M E, Olson J A, Shike M. Modem Nutrition in Health and Disease, Philadelphia, Lea & Febiger, 8^{th} ed., Chapter 33, 1994). Whey protein has more cysteine (and tyrosine) than eggs and soy protein. Cysteine can de-toxify soluble and lipid peroxidases, which are prevalent during stress. The peroxidases damage the cell membranes.

Research has shown that the amino acid glutamine improves immune function. For example, glutamine (14 g), given during 14 days of training in the Army's Special Forces Assessment and Selection Course, increased the percentage (50% vs. 30%) of soldiers who had an induration >10 mm in response to tetanus toxoid compared to those receiving glycine, (R. L. Shippee, et al. pages 90-93 In: Ross Conference on Medical Research, Nutritional Immunomodulation in Disease and Health Promotion, Columbus: Ross Products Division, 1996).

Choline

Although choline is found in free form in nature, it has no known functions except as a constituent of larger molecules. These larger molecules include the neurotransmitter acetylcholine, the cell membrane phospholipid, phosphatidylcholine, and the neural membrane phospholipid sphingomyelin. Choline is also a constituent of both platelet activating factor and plasmalogen and pulmonary surfactant. These choline containing molecules are involved in a wide range of biological functions, so it is not surprising that choline deficiency in animals is associated with wide sweeping effects, including renal dysfunction, growth impairment and memory impairment.

The choline-containing molecule sphingomyelin is a component of neural membranes. Consumption of choline can raise plasma choline levels, resulting in an increase in brain choline and acetylcholine levels. In a test involving college students as subjects, 25 grams of phosphatidylcholine was shown to improve explicit memory (as measured by a serial learning task) 90 minutes after ingestion (Clinical Neuropharmacology, 16:540 (1993)).

Choline supplementation appears to reduce carnitine excretion in the urine that leads to an increased capacity of the tissues to oxidize fatty acids. During exercise, choline concentrations decline; taking extra choline prior to exercise or an exhausting day may prevent this decline and thus improve performance (Ultralec™, Technical Report, ADM Inc., Decatur, Ill., 1998). ADM has recently developed a de-oiled form of choline from lecithin called Ultralec™. It is in the form of light brown granules or powder.

Diets low in choline have been shown to reduce muscle performance. Choline has also been shown to enhance memory and reaction time. Most people take in at least 200 to 1,000 mg of choline per day. The dietary supplement can include approximately 500 mg, in a preferred embodiment.

Creatine

Creatine is found in skeletal muscle, heart, all smooth muscle tissue, sperm and neural tissue. It is synthesized in the liver and kidney and released into the blood. Three amino acids are required for its synthesis: glycine, arginine and methionine (Clark, Nutrition, 14:321-333, 1998). Research shown that supplementation of 2 to 5 g per day of creatine increases muscle creatine content. In the muscle, creatine is reversibly converted to phosphocreatine (PCr) by the enzyme creatine kinase (CK). The pool of PCr is considered a high-energy phosphate buffer of ATP and a larger energy reserve than ATP. Athletes who take about 20 g of creatine during exercise have a rapid resynthesis of PCr afterwards. This is due to the ability of creatine to stimulate the mitochondria, which results in increased anaerobic capacity and aerobic recovery. (J. F. Clark, Nutrition, 14:322-324 (1998)). Muscle growth is also enhanced.

A typical dose of creatine for athletes is 20 g per day for 1 to 2 weeks followed by a reduction in the dose to 5 g per day. Higher doses appear to have little benefit. Males and females given 20 g per day of creatine had increased lean body mass determined by DEXA, and no change in body fat, blood pressure, and plasma creatine kinase (Mibie S., “The effect of creatine supplementation on blood pressure, plasma creatine kinase, and body composition.” FASEB, 1998, abstract #3791). The dietary supplement can be formulated to supply, in a daily intake, a starting amount of creatine (e.g., about 20 grams) or a maintenance amount of creatine (e.g., about 5 grams).

In one study, creatine monohydrate (20 g per day for five days and 3 g daily for the remaining 9 weeks of the study) was given to male college football players. All subjects were enrolled in a weight training programs. Fat free mass increased significantly (4.4%) in the creatine group. Total body water increased 5% and intracellular water increased 3.2% in the creatine group with no changes in the placebo group. There was also a significant increase in strength in the treatment group as determined by improvement in bench press and squat exercises (Knehane A., “Creatine supplementation affects body composition and neuromuscular performance in football athletes.” FASEB, 1998, abstract #4994)

In another study, twenty-five soldiers were randomly assigned to receive 24 g of creatine monohydrate in a sports bar or a similar bar without the creatine. The treatment group had a significant (14.4%) increase in total repetitions over five sets of bench presses. The time to complete an obstacle course was (not significantly) reduced by 7.6±8.2 seconds vs. 5.9±12 seconds in the control group. Body mass increased by 1.5 kg and body fat decreased by 0.6% (p <0.05) in the creatine group. (J. P. Warber et al., “Effects of creatine monohydrate supplementation on physical performance.” FASEB, 1998, abstract #6016)

Embodiments of the Invention

Servings of the dietary supplement may vary in size and are not limited to units supplying the sum of the weights of the ingredients for the recipe or the preferred daily intake of nutrients listed in the third column of Table 2. A dietary supplement which supplies, in a recommended daily intake, nutrients comprising those listed in Table 2, can be ingested in various amounts throughout a given day, and the term “dietary supplement” is not intended to be limited to a particular weight or dose of the dietary supplement. It is understood by those of skill in the art that other ingredients can be added to those listed in Table 2, for example, fillers, emulsifiers, preservatives, etc. for the processing or manufacture of a food product.

TABLE 2

| | | |
|---|---|---|
| | | Daily Intake |
| Nutrients per bar | Optimal Recipe | (preferred range) |

| | | | |
|---|---|---|---|
| Carbohydrate | 15 | g | 1-60 g (10-30 g) |
| Protein | 5 | g | 1-40 g (3-10 g) |
| Nuts | 10 | g | 1-100 g (20-30 g) |
| Fish Oil | 5 | g | 1-20 g (5-15 g) |
| MCT Oil | 3 | g | 1-40 g (5-10 g) |
| Borage Oil | 4.5 | g | 1-40 g (3-10 g) |
| Choline | 500 | mg | 1-2,000 mg (250-750 mg) |
| Glutamine | 15 | g | 1-50 g(5-20 g) |
| Creatine | 20 | g | 1-30 g (5-25 g) |

Recommended Range of Ingredients for Daily Intake (See Third Column of Table 2)

1-100 g nuts, e.g., walnuts, peanuts, cashews, pistachios, Brazil nuts, hazelnuts, pecans, almonds, etc. (preferred 20-30 g per day)

1-20 g fish oil from menhaden, salmon, for instance, or vegetable oils such as flaxseed oil, evening primrose oil, soybean oil, canola oil, olive oil (preferred 5-15 g per day)

1-40 g medium-chain triglycerides from coconut, palm oil, or palm kernel oil, for example (preferred 5-10 g per day)

carbohydrate: simple carbohydrates, oligosaccharides, complex carbohydrate

1-20 g sucrose (as the preferred simple carbohydrate, 5-10 g per day preferred)

1-20 g maltodextrins from corn syrup solids (as the preferred oligosaccharide, 5-10 g per day preferred)

1-20 g uncooked cornstarch (as the preferred complex carbohydrate, 5-10 g per day preferred)

1-40 g protein from, for example, whey, casein, milk, eggs, soy (preferred 3-10 g per day)

1-40 g borage oil (preferred 3-10 g per day)

1-2,000 mg choline, preferably from lecithin preferred 250 to 750 mg per day)

1-50 g glutamine (preferred 5-20 g per day; can be in the form of the sodium or potassium salt, for example)

1-30 g creatine (preferred 5-25 g per day)

0-600 mg caffeine (preferred 250 to 300 mg/day; optional)

0-2 g carnitine (preferred 50 to 100 mg/day; optional)

Selected Compositions of Dietary Supplement Ingredients

10 g walnuts supply 63 kcal as 1.4 g protein, 6.8 g fat and 0.3 g carbohydrate.

5 g fish oil supply 45 kcal as 5 g fat.

3 g medium-chain triglycerides supply 25 g kcal as 3 g fat.

15 g carbohydrate (as 5 g simple carbohydrate, 5 g maltodextrin, and 5 g uncooked cornstarch in a preferred embodiment) supply 60 kcal as 15 g carbohydrate.

5 g protein (from whey or soy or from a blend of the two in a preferred embodiment) supply 20 kcal as 5 g protein.

500 mg choline as Ultralec™ (ADM, Decatur, Ill.). Ultralec P, a preferred source of choline, is approximately 3.6% choline. Thus, 13.8 g would be necessary to supply a preferred amount of 500 mg of choline.

By one analysis, the fatty acid composition of walnuts per 100 g is:

Total fat=61.2 g

Saturated, total=7.6 g as palmitic 5.4 g; 2.2 g as stearic; and 0.1 as arachidonic;

Monounsaturated, total=11.0 g as 10.8 g as oleic; 0.2 g as gadoleic;

Polyunsaturated, total=42.6 g as 35.2 g linoleic; 7.4 g as linolenic;

Ratio of omega 6/omega 3=4.76

Further Ingredients

The dietary supplement can also contain other ingredients in addition to those listed in Table 2, such as one or a combination of other vitamins, minerals, antioxidants, fiber and other dietary supplements. Selection of one or several of these ingredient is a matter of formulation, design, consumer preference and end-user. The amount of these ingredients added to the dietary supplements of this invention are readily known to the skilled artisan and guidance to such amounts can be provided by the U.S. RDA doses for children and adults. Vitamins and minerals that can be added include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D₃; cyanocobalamin; sodium selenite; copper sulfate; vitamin A; vitamin B₆ and hydrochloride thereof; vitamin C; inositol; vitamin B₁₂; potassium iodide.

Flavors, coloring agents, spices, nuts and the like can be incorporated into the product. Flavorings can be in the form of flavored extracts, volatile oils, chocolate flavorings, peanut butter flavoring, cookie crumbs, crisp rice, vanilla or any commercially available flavoring. Examples of useful flavoring include but are not limited to pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In a preferred embodiment, the dietary supplement contains cocoa or chocolate.

Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product.

Preservatives may also be added to the dietary supplement to extend product shelf life. Preferably, preservatives such as potassium sorbate, sodium sorbate, potassium benzoate, sodium benzoate or calcium disodium EDTA are used.

In addition to the carbohydrates described above, the nutritional supplement can contain artificial (preferably low calorie) sweeteners, e.g., saccharides, cyclamates, aspartamine, aspartame, acesulfame K, and/or sorbitol. Such artificial sweeteners can be desirable if the dietary supplement is intended to be consumed by an overweight or obese individual, or an individual with type II diabetes who is prone to hyperglycemia.

The dietary supplement can be provided in a variety of forms, so long as the cornstarch is not heated to the point of significant hydrolysis. These forms can include beverages, baked goods, wherein the cornstarch is in a portion that is not baked, puddings, confections, snack foods, or frozen confections or novelties.

In a preferred embodiment, to manufacture a food bar, the liquid ingredients are cooked; the dry ingredients are added with the liquid ingredients in a mixer and mixed until the dough phase is reached; the dough is put into an extruder, and extruded; the extruded dough is cut into appropriate lengths; and the product is cooled. The bars may contain other nutrients and fillers to enhance taste, in addition to the ingredients specifically listed herein. Any portion of the nutritional supplement containing uncooked cornstarch cannot be baked because the uncooked cornstarch will break down and then will not provide a delayed release of sugar into the blood. Thus, the bars can be extruded—much like candy is made.

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

What is claimed is:

1. A dietary supplement comprising nuts, creatine, glutamine, high biological value protein, one or more oils containing omega-3 fatty acids, medium chain triglycerides, borage oil, choline, and a combination of carbohydrates comprising one or more simple carbohydrates, one or more oligosaccharides, and one or more complex carbohydrates.

2. The dietary supplement of claim 1, wherein the nuts are walnuts.

3. The dietary supplement of claim 1 wherein the nutritional supplement is in the form of an extruded bar.

4. The dietary supplement of claim 1 further comprising caffeine.

5. The dietary supplement of claim 1 further comprising carnitine.

6. The dietary supplement of claim 1 wherein the simple carbohydrate comprises sucrose.

7. The dietary supplement of claim 1 wherein the oligosaccharide comprises maltodextrin.

8. The dietary supplement of claim 1 wherein the complex carbohydrate comprises uncooked cornstarch.

9. The dietary supplement of claim 1 wherein the choline is supplied by lecithin.

10. The dietary supplement of claim 1 wherein the oils containing omega-3 fatty acids comprise flaxseed oil.

11. The dietary supplement of claim 1 further comprising flaxseed oil.

12. A dietary supplement which supplies, in a recommended daily intake, nutrients comprising from about 1 to about 60 grams carbohydrate, from about 1 to about 40 grams high biological value protein, from about 1 to about 100 grams nuts, from about 1 to about 20 grams fish oil or vegetable oil high in omega-3 fatty acids, from about 1 to about 40 grams medium-chain triglycerides, from about 1 to about 40 grams borage oil, from about 1 to about 2,000 milligrams choline, from about 1 to about 50 grams glutamine, and from about 1 to about 30 grams creatine.

13. The dietary supplement of claim 12 which supplies, in a recommended daily intake, from about 10 to about 30 grams carbohydrate.

14. The dietary supplement of claim 12 which supplies, in a recommended daily intake, from about 3 to about 10 grams high biological value protein.

15. The dietary supplement of claim 12 which supplies, in a recommended daily intake, from about 20 to about 30 grams nuts.

16. The dietary supplement of claim 12 which supplies, in a recommended daily intake, from about 5 to about 15 grams fish oil or vegetable oil high in omega-3 fatty acids.

17. The dietary supplement of claim 12 which supplies, in a recommended daily intake, from about 5 to about 10 grams medium-chain triglycerides.

18. The dietary supplement of claim 12 which supplies, in a recommended daily intake, from about 3 to about 10 grams borage oil.

19. The dietary supplement of claim 12 which supplies, in a recommended daily intake, from about 250 to about 750 milligrams choline.

20. The dietary supplement of claim 12 which supplies, in a recommended daily intake, from about 5 to about 20 grams glutamine.

21. The dietary supplement of claim 12 which supplies, in a recommended daily intake, from about 5 to about 25 grams creatine.

22. The dietary supplement of claim 12 wherein the carbohydrate comprises a carbohydrate having a high glycemic index, a carbohydrate having an intermediate glycemic index, and a carbohydrate having a low glycemic index.

23. The dietary supplement of claim 22 wherein the carbohydrate having a high glycemic index, the carbohydrate having an intermediate glycemic index, and the carbohydrate having a low glycemic index are in approximately equal amounts by weight.

24. The dietary supplement of claim 12 wherein the complex carbohydrate comprises uncooked cornstarch.

25. The dietary supplement of claim 12 wherein the high biological value protein comprises at least one protein source selected from the group consisting of: whey powder, egg protein, soy protein, soy protein isolate, sodium caseinate, sodium calcium caseinate, calcium caseinate, and potassium caseinate.

26. A dietary supplement which supplies, in a recommended daily intake, nutrients comprising from about 13 to about 60 grams carbohydrate, from about 3 to about 10 grams high biological value protein, from about 20 to about 30 grams nuts, from about 5 to about 15 grams fish oil or vegetable oil high in omega-3 fatty acids, from about 5 to about 10 grams medium-chain triglycerides, from about 3 to about 10 grams borage oil, from about 250 to about 750 milligrams choline, from about 5 to about 20 grams glutamine, and from about 5 to about 25 grams creatine.

27. A method for providing an individual with dietary supplementation that alleviates the effects of stress, comprising administering to the individual a dietary supplement comprising nuts, creatine, glutamine, high biological value protein, one or more oils containing omega-3 fatty acids, medium chain triglycerides, borage oil, choline, and a combination of carbohydrates comprising simple carbohydrate, oligosaccharide, and complex carbohydrate.

28. A method for providing an individual with dietary supplementation that alleviates the effects of stress, comprising administering to the individual a dietary supplement which supplies, in a recommended daily intake, nutrients comprising from about 1 to about 60 grams carbohydrate, from about 1 to about 40 grams high biological value protein, from about 1 to about 100 grams nuts, from about 1 to about 20 grams fish oil or vegetable oil high in omega-3 fatty acids, from about 1 to about 40 grams medium-chain triglycerides, from about 1 to about 40 grams borage oil, from about 1 to about 2,000 milligrams choline, from about 1 to about 50 grams glutamine, and from about 1 to about 30 grams creatine.

29. The method of claim 28 wherein the dietary supplement is administered in two approximately equal doses which each provide approximately half of a recommended daily intake.

30. The method of claim 28 wherein the dietary supplement is administered as a food bar.
